(19) United States
(12) Patent Application Publication
(10) Pub. No.: US 2013/0250242 A1
(43) Pub. Date: Sep. 26, 2013

(54) FUNDUS CAMERA

(71) Applicant: OPTOVUE, INC., Fremont, CA (US)

(72) Inventors: Yeou-Yen Cheng, Saratoga, CA (US); Jay Wei, Fremont, CA (US)

(21) Appl. No.: 13/830,113

(22) Filed: Mar. 14, 2013

Related U.S. Application Data
(60) Provisional application No. 61/613,919, filed on Mar. 21, 2012.

(51) Int. Cl. A61B 3/14; A61B 3/15

(52) U.S. Cl. CPC A61B 3/14; A61B 3/156
USPC 351/207; 351/206; 351/246

(57) ABSTRACT

An apparatus and method of providing an ophthalmic image is presented. In accordance with embodiments of the present invention, an illumination path with a focus index optical assembly and a first diopter compensator is provided and an imaging path with a sensor and a second diopter compensator is provided. The first diopter compensator and the second diopter compensator can be adjusted to provide focus according to a focus index from the focus index optical assembly.

### RELATED APPLICATIONS

This application claims priority to U.S. Provisional Application 61/613,919, filed on Mar. 21, 2012, which is herein incorporated by reference in its entirety.

### BACKGROUND

1. Field of the Invention

Embodiments of the present invention are related to an ophthalmic photographing apparatus.

2. Discussion of Related Art

In a conventional fundus camera, a focus index, such as a split-bar pattern as, for example, shown in FIGS. 1A-1C, is usually generated from a focus index projection system using a light source with wavelength in the range of dark red to Near InfraRed (NIR) illuminating a slit. The focus index projection system is then branched into the fundus illumination path through a beam splitter or a flipping mirror (shutter) (see, for example, U.S. Pat. No. 3,925,793 A and U.S. Pat. No. 4,283,124 A). Another way of branching the focus index path into the fundus illumination path is through the projection of the focus index on to a retractable stick (or spot) mirror which is conjugate to the fundus (see, for example, U.S. Pat. No. 4,412,728 A and U.S. Pat. No. 4,400,070 A). The split-bar pattern is then re-imaged at the fundus of the eye under examination after the illumination beam passes through the ocular lens and the eye. The video image of the fundus, superimposed with the focus index for observation or alignment purpose, passes through the eye, the ocular lens, the aperture, and the imaging lens system before it is received by the image sensor and then can be displayed on the monitor (not shown). FIG. 1A illustrates split-bar pattern 30 in focus while FIGS. 1B and 1C illustrate split-bar pattern 30 when out of focus.

The operator judges the degree of focus by assessing the alignment of the two halves of the split-bar image/focus pattern 30, as illustrated in FIGS. 2A-2C displayed on a monitor image 32. When the focus is correct, the two halves of the split bar image 30 become aligned as shown in FIG. 2A. Otherwise, the split-bar pattern 30 is misaligned and the two bars separated from each other, depending on the direction and amount of defocus as illustrated in FIG. 2B and FIG. 2C. After the operator is finished focusing by aligning the focus index and triggered an image acquisition, a control system of the fundus camera turns off the NIR light sources for both the fundus and the focus index illumination and retracts the retractable stick mirror out of the main illumination path before turning on a flash light (white light) for illumination to capture a color fundus image (still image).

Due to engineering limitations of the system design, the total diopter range for continuous focus adjustment (by either moving the focus lens or the sensor) of a conventional fundus camera is typically less than 30 diopters and is not enough for patients with severe ametropia. Therefore, when examining a patient with severe ametropia, a Diopter Compensation (DC) lens is usually inserted in the imaging path of the conventional fundus camera, either manually or automatically, to perform diopter correction. In this way, the nominal diopter range of the camera can be extended to cover a wider diopter range to capture fundus image which might not be at the optimal focus. However, the moving range of the focus index subassembly is limited compared to that of the focus adjustment of the imaging path when the DC lens is used.

An example of a conventional imager is illustrated in FIGS. 3A through 3C. As shown in FIG. 3A, light is incident on relay lens 13. Relay lens 13 images a light source on eye 100 and can serve as the collimating lens for a focus index illuminating light beam. Light from relay lens 13 is then input on focus index assembly 9, which can be moved over a diopter range. Focus index assembly 9 forms a split-bar image as illustrated in FIGS. 1A-1C. Light from focus index assembly 9 is incident on dot plate 21. Dot plate 21 is commonly used in a fundus camera to eliminate surface reflections from an ocular lens 1. Light from dot plate 21 is directed through folding mirror 7, lens group 6, cornea diaphragm 17, perforated mirror 2, and ocular lens 1 to be incident on eye 100. Reflections from eye 100 pass through perforated mirror 2 and aperture stop 3 to be incident on focus lens 4a. Focus lens 4a can be moved over a diopter range. Light from focus lens 4a is then input to relay lens system 4 before being incident on sensor 5.

In FIG. 3A, both the focus lens 4a and the focus index assembly 9 can be moved continuously within the diopter range from −10 D to +10 D. However, in FIG. 3B, when a negative DC lens 22b is inserted between aperture stop 3 and focus lens 4a, the diopter range extends to a range of −25 D to −5 D in the imaging path, while the range in the illumination path is still limited to a range of −10 D to +10 D. Similarly, in FIG. 3C, when a positive DC lens 22c is inserted into the imaging path between aperture stop 3 and focus lens 4a, the diopter range of the imaging path extends to a range of +5 D to +25 D, while the illumination path stays in −10 D to +10 D diopter range.

FIGS. 4A, 4B, and 4C illustrate split-bar 30 for focusing in a nominal range eye after fine focusing the camera with no DC lens as shown in FIG. 3A. FIG. 4B illustrates split-bar 30 for an eye with severe myopia after rough focusing with the negative DC lens 22b in the imaging path as shown in FIG. 3B. FIG. 4C illustrates an eye with severe hyperopia after rough focusing with a positive DC lens 22c as shown in FIG. 3C.

Since the diopter range may not be compatible with that of the imaging path, the focus index is not able to provide indications for accurate focus adjustment (FIGS. 4B and 4C) and therefore, the focus index subassembly 9 is commonly withdrawn from the illumination path to reduce confusion while the operator performs focus adjustment without the aid of the focus index. Therefore, continuous fine focus adjustment is not possible in the case of severe ametropia.

For the case using a non-mydriatic fundus camera to examine a severe ametropic eye, where Near Infrared (NIR) light source was used during observation, the quality of a captured color fundus image is much worse because the focus shifts between the NIR video image in the observation mode and the captured color image (still image). One method of solving this problem was disclosed in the U.S. Pat. No. 7,837,329 B2, where a larger space between the Polarization Beam-Splitter (PBS) and the focus index subassembly was reserved to accommodate greater moving range for the focus index subassembly when the DC lens was inserted into the imaging path. However, this approach is not suitable for compact system design where space is very limited. Another method disclosed in the same patent was that additional correction lenses were inserted into the space between the PBS and the focus index subassembly to reduce the amount of movement of the focus index subassembly. However, this approach requires a strong power correction lens with large diopter scale, and inadvertently increases sensitivity to component tolerances and alignment error.

Therefore, a system and method for better continuous fine-focus adjustment using an extended diopter range for both the imaging and the illumination path is needed.

### SUMMARY

In accordance with some embodiments of the present invention, an ophthalmic imaging apparatus is presented. An ophthalmic imaging apparatus according to some embodiments of the present invention can include an illumination path; an imaging path; a sensor in the imaging path; a focus index optical assembly in the illumination path; a first diopter compensator in the illumination path; and a second diopter compensator in the imaging path, the first diopter compensator and the second diopter compensator cooperating to provide a focused image at the sensor.

A method of providing an ophthalmic image according to some embodiments of the present invention includes providing an illumination path with a focus index optical assembly and a first diopter compensator; providing an imaging path with a sensor and a second diopter compensator; and adjusting the first diopter compensator and the second diopter compensator to provide focus according to a focus index from the focus index optical assembly.

These and other embodiments are further described below with respect to the following figures.

### BRIEF DESCRIPTION OF THE DRAWINGS

FIG. 1A shows an exemplary split-bar pattern when focus is achieved.

FIG. 1B shows an exemplary split-bar pattern with a positive focus error.

FIG. 1C shows an exemplary split-bar pattern with a negative focus error.

FIG. 2A shows an exemplary split-bar pattern superimposed on a fundus image when focus is achieved.

FIG. 2B shows an exemplary split-bar pattern superimposed on a fundus image with a positive focus error.

FIG. 2C shows an exemplary split-bar pattern superimposed on a fundus image with a negative focus error. FIG. 3A shows exemplary optical schematics of a fundus camera with movable focus lens showing the diopter range without a DC lens.

FIG. 3B shows exemplary optical schematics of a fundus camera with movable focus lens showing the diopter range with a negative DC lens in the imaging path.

FIG. 3C shows exemplary optical schematics of a fundus camera with movable focus lens showing the diopter range with a positive DC lens in the imaging path.

FIGS. 4A, 4B, and 4C show a split-bar pattern superimposed on a fundus image corresponding to each individual configuration of FIGS. 3A, 3B, and, 3C, respectively, when focus adjustment is performed.

FIG. 5A shows an exemplary optical schematic with a movable sensor showing diopter range corresponding to the movement of the sensor and the focus index subassembly.

FIG. 5B shows an exemplary optical schematic with a movable sensor showing matching diopter range of the sensor and the focus index subassembly with a negative DC lens in both the imaging and the illumination path.

FIG. 5C shows an exemplary optical schematic with a movable sensor showing matching diopter range of the sensor and the focus index subassembly with a positive DC lens in both the imaging and the illumination path.

FIG. 6A shows an aligned split-bar pattern superimposed on a fundus image when focus is achieved with no DC lens.

FIG. 6B shows an aligned split-bar pattern superimposed on a fundus image with a negative DC lens inserted in both the imaging and the illumination paths for severe myopia patients.

FIG. 6C shows an aligned split-bar pattern superimposed on a fundus image with positive DC lens inserted in both the imaging and the illumination path for severe hyperopia patients.

FIG. 7 is an exemplary optical schematic of a fundus camera showing DC lenses in both the imaging and the illumination paths with a movable focus lens.

FIG. 8 is an exemplary optical schematic of a fundus camera showing DC lenses in both the imaging and the illumination paths with a movable sensor.

FIGS. 9A and 9B show an example of a focus index optical assembly with multiple fixation targets.

FIGS. 10A and 10B show a lens slider mounted with different compensation lenses for the imaging path in some embodiments.

FIGS. 11A and 11B show a lens slider mounted with different compensation lenses for the illumination path in some embodiments.

FIG. 12 shows an exemplary monolithic DC lens slider in some embodiments.

FIG. 13 is an exemplary optical schematic of the fundus camera with tunable lenses as the DC lenses in both the imaging and the illumination paths with three dot plates in some embodiments.

FIG. 14 is an exemplary optical schematic of FIG. 13 with the cornea diaphragm in a different location.

FIG. 15 is an exemplary optical schematic of FIG. 13 with two dot plates.

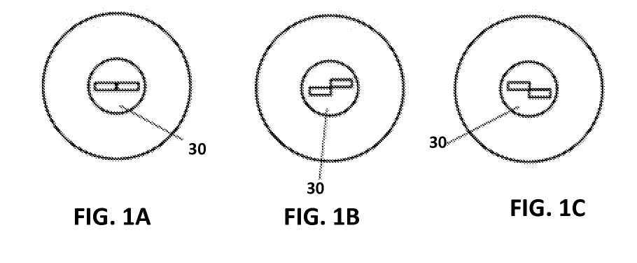

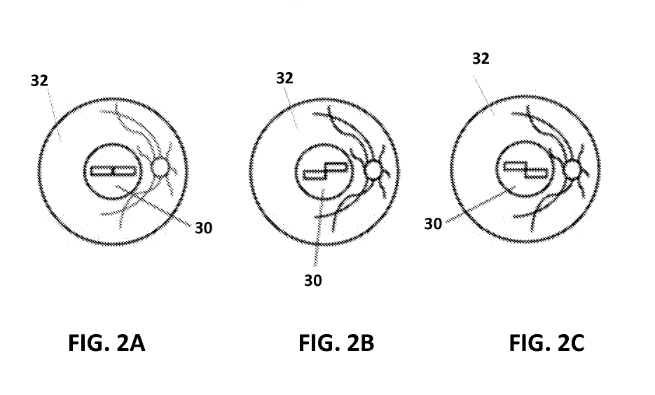

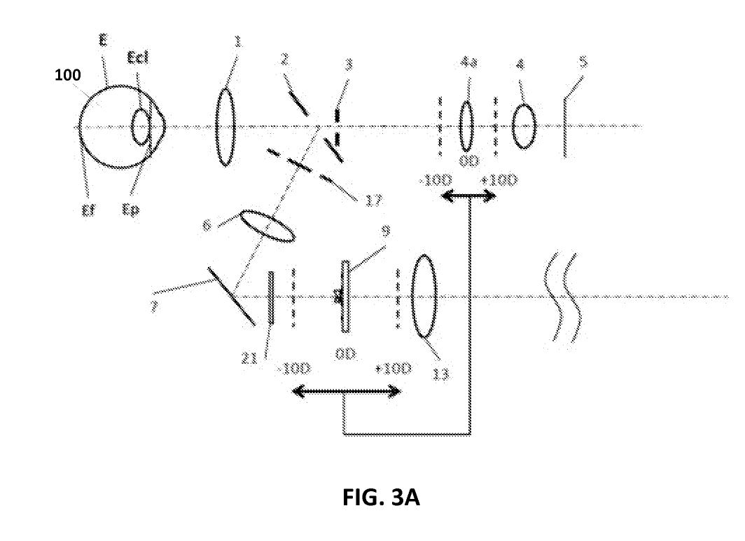

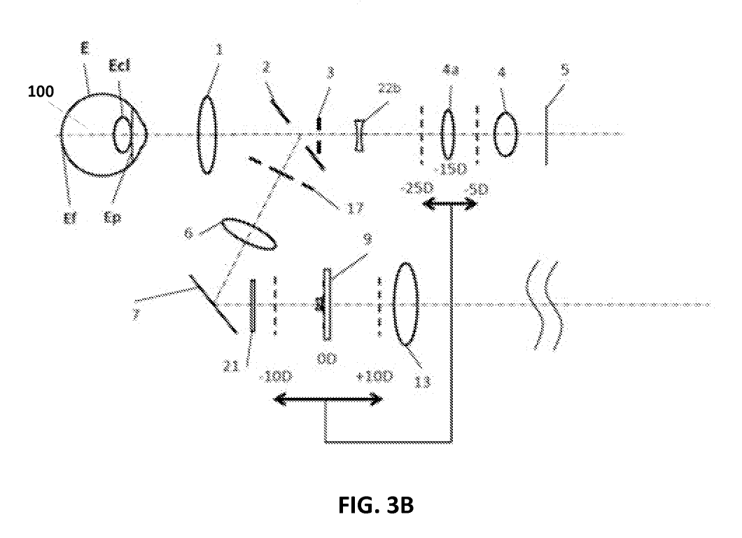

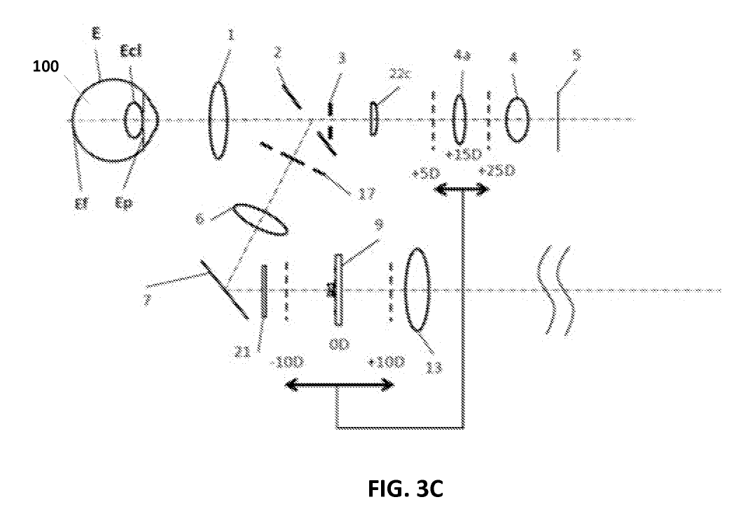

### DETAILED DESCRIPTION

Embodiments of the present invention are described herein with reference to the exemplary drawings.

According to some embodiments of the present invention, a system and method of changing the illumination path to match the amount of diopter variation in the imaging path with a DC lens is disclosed. This allows continuous fine-focus adjustment using the focus index 30 (the split-bar) to be achieved without increase in size of the optical system. In some embodiments, a tunable lens can be used individually in either the imaging or the illumination path. Or a tunable lens can be used simultaneously in both the illumination path and the imaging path to allow continuous fine-focus adjustment.

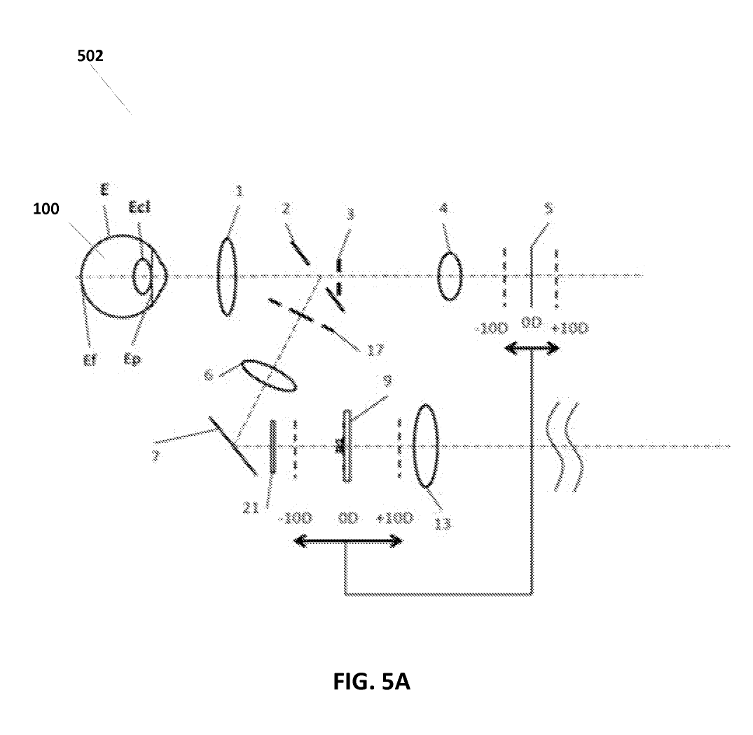

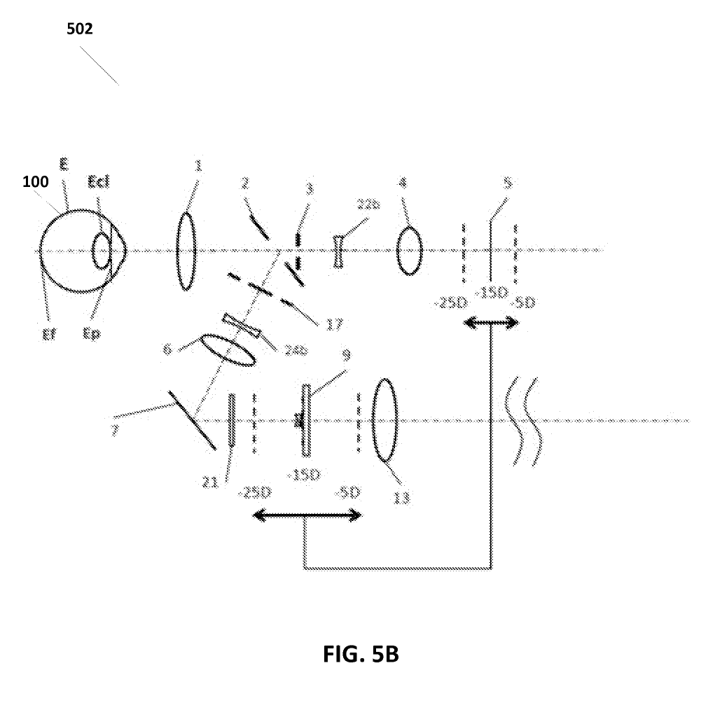

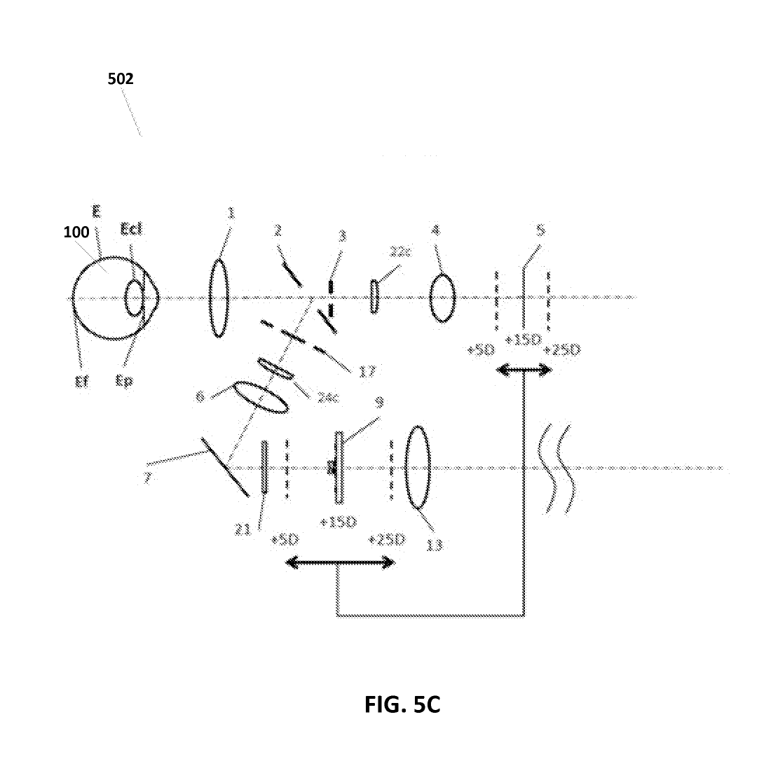

FIG. 5A shows an exemplary schematic of a fundus camera 502 according to some embodiments where sensor 5 is movable. In FIG. 5B, a DC lens 22b is inserted into the imaging path and another DC lens 24b is also inserted into the illumination path between the cornea diaphragm 17 and the last surface of the relay lens system 6. In some embodiments, the DC lens 22b and the DC lens 24b can have equivalent or similar optical power. In the embodiment of camera 502 illustrated in FIG. 5B, when the negative DC lens 22b of the imaging path is inserted in the optical path, a negative DC lens 24b with matching power can also be inserted into the illumination path to shift the focus range of the focus index toward a more negative diopter range, such as from −25 D to −5 D, as compared to the range of −10 D to +10 D illustrated in FIG. 5A.

Similarly, in the embodiment of fundus camera 502 illustrated in FIG. 5C a DC lens 24c with positive power can be used to shift the focus range of the focus index toward a more positive diopter range, such as +5 D to +25 D. Using this approach, continuous fine-focus adjustment using the focus index 30 for patients with severe myopia or hyperopia can still be achieved.

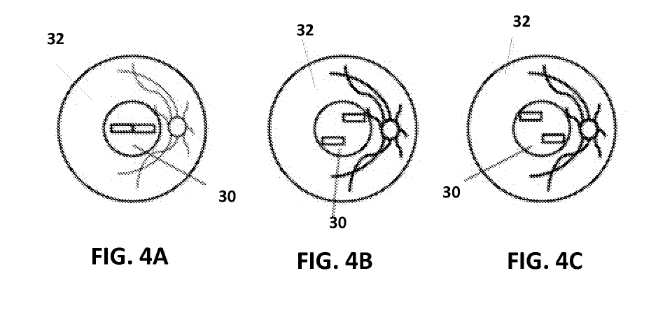

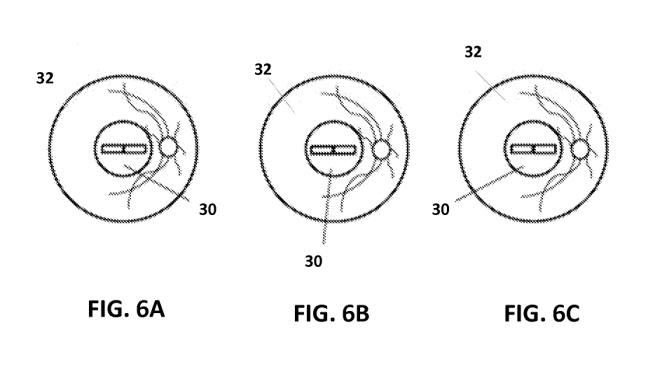

FIGS. 6A-6C illustrate the ability to focus using fundus camera 502 as illustrated in 5A-5C, respectively. As shown in FIG. 6A, an aligned focus index 30 in a normal range eye can be achieved without a DC lens. However, as opposed to the focus illustrated in FIG. 4B, FIG. 6B illustrates an aligned focus index 30 using fundus camera 502 on an eye with severe myopia where negative DC lenses are inserted in both the imaging and illumination paths (negative DC lens 22b and negative DC lens 24b as illustrated in FIG. 5B). Further, as opposed to the focus illustrated in FIG. 4C, FIG. 6C illustrates an aligned focus index 30 using fundus camera 502 on an eye with sever hyperopia where positive DC lenses are inserted in both the imaging and illumination paths (positive DC lens 22c and positive DC lens 24c as illustrated in FIG. 5C).

In FIGS. 4A and 6A, the patient eye is within a normal range and fine-focus can be performed (split-bar focus index 30 aligned) without the use of the DC lens. In FIG. 4B, fine-focus was not possible because the negative DC lens is only used in the imaging path, while FIG. 6B shows the results of fine-focus with the negative DC lens inserted in both the imaging path and the illumination path in accordance with some embodiments. Similarly, in FIG. 4C, fine-focus was also not possible because the positive DC lens is only used in the imaging path, while FIG. 6C shows the results of fine-focus with the positive DC lens inserted in both the imaging path and the illumination path in some embodiments.

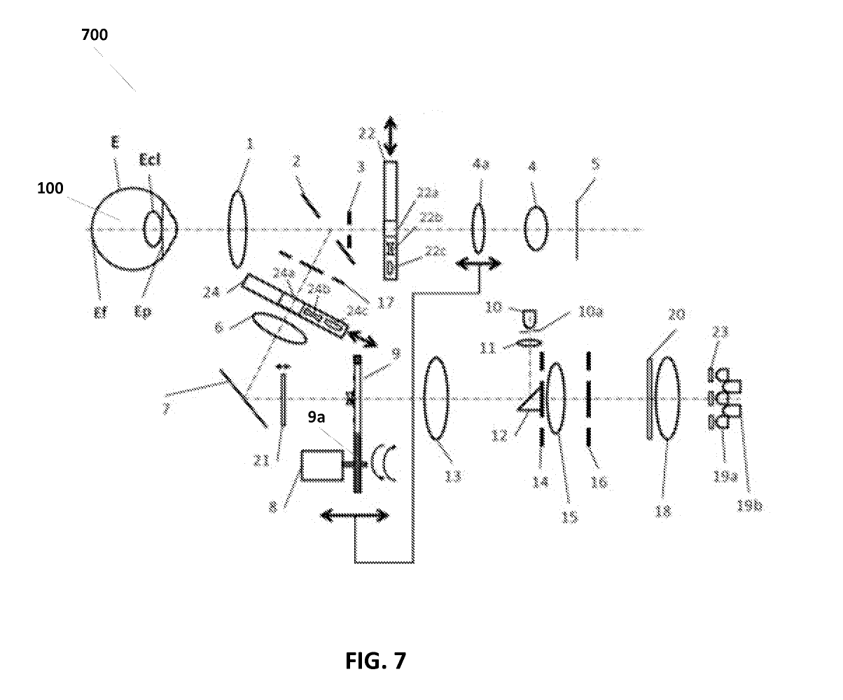

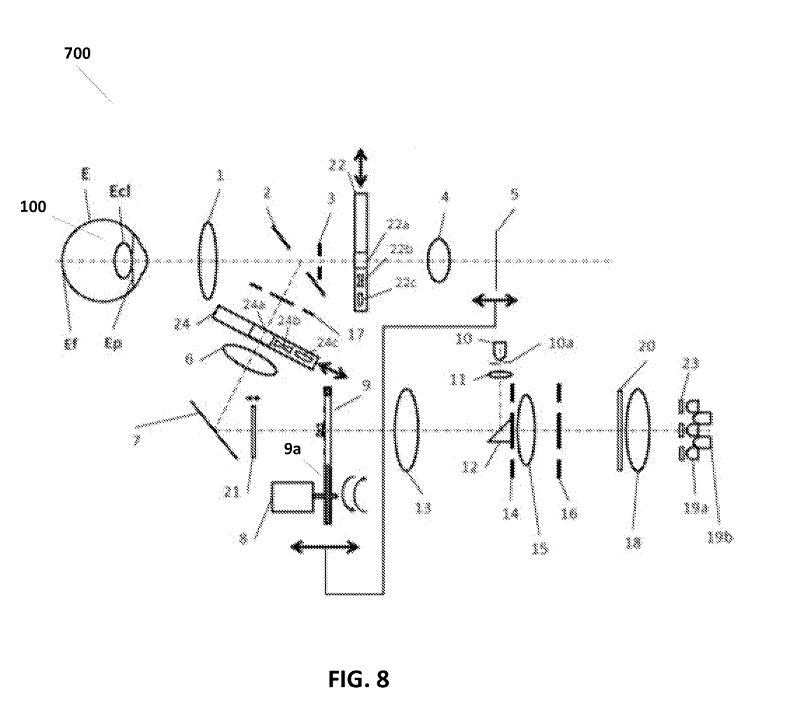

FIG. 7 and FIG. 8 show exemplary schematics of a fundus camera 700 in accordance with some embodiments of the present invention. As shown in FIG. 7, sensor 5, which can be a multi-band detector, is fixed and lens 4a can be moved along the optical axis of the imaging path for focusing. In the embodiment illustrated in FIG. 8, lens 4a is fixed and sensor 5 can be moved to focus the image. In the embodiments illustrated in FIG. 7 and FIG. 8, diopter compensators such as a DC lens slider 24 and DC lens slider 22 can be positioned in camera 700. A DC lens slider 24 that can position DC lens 24a, negative DC lens 24b, or positive DC lens 24c in the illumination path is provided. Additionally, a DC lens slider 22 that can position DC lens 22a, negative DC lens 22b, and positive DC lens 22c within the image path is provided. DC lens 24a and DC lens 22a represent the absence of a DC lens.

The focus index illuminating light source 10, which can be, for example, a NIR LED, is mounted on a fixed part (not shown). Such fixed part can be a lens housing mounted on a base structure of camera 700. In some embodiments, this fixed part is kept further away from the movable focus index optical assembly 9 to minimize the vibration or shock energy by-product generated from the rapid in and out retraction motion of the focus index optical assembly 9 during each switching cycle between the observation mode and the image acquisition mode. Therefore the reliability of the focus index illumination can be improved by reducing the vibration and shock by-product. The black dot plate 21 is commonly used in a fundus camera to eliminate surface reflection of the ocular lens 1. In FIG. 7 and FIG. 8, the black dot plate 21 can be moved when DC lens slider 24 positions different lenses 24a, 24b, or 24c in the illumination beam to continue to eliminate the unwanted surface reflection.

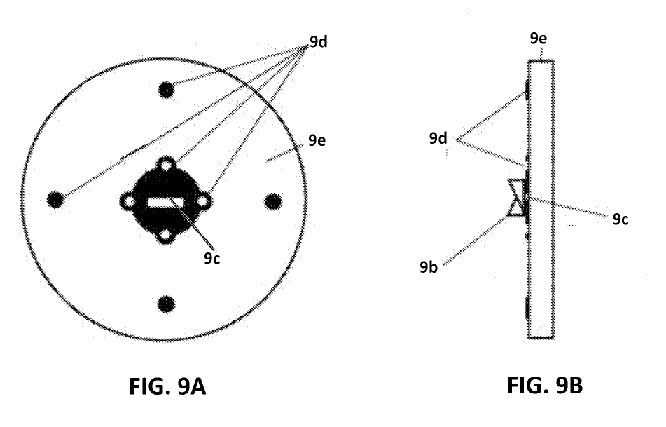

In some embodiments, a field stop 10a is attached in front of light source 10, such as a NIR LED, as shown in FIG. 7 and FIG. 8, so that it is re-imaged by the relay lens 11 to a position near the front focal plane of the second relay lens 13 of the fundus illumination path. The second relay lens 13 re-images the crystalline lens diaphragm 14 to a surface close to the back surface of the crystalline lens (Ecl) of eye 100. In this arrangement, the relay lens 13 also serves as the collimating lens for the focus index illuminating light beam generated from the light source 10. A small folding mirror 12, such as a prism mirror, can be attached onto and hide behind the central disk of the crystalline lens diaphragm 14 to minimize interference with the fundus illumination beam passing through the ring opening of the diaphragm 14. After being reflected by the small folding mirror 12, the focus index illumination beam illuminates the focus-index optical subassembly 9 and form a split-bar image on the eye fundus (Ef) of eye 100 through folding mirror 7, relay lens group 6, DC lens slider 24, cornea diaphragm 17, perforated mirror 2, ocular lens 1, and the eye 100. An embodiment of index optical subassembly 9 is discussed in further detail with respect to FIGS. 9A and 9B below.

In the fundus observation mode, the illumination is achieved by turning on the NIR LED ring array 19b of a dual band interlaced LED ring array 19a and 19b and the focus index illuminating light source 10, as shown in FIGS. 7 and 8. The NIR light generated from the array 19b is focused on the ring aperture 16 through the opening of the mount, such as a PCB (Printed Circuit Board) of the white LED array 19a, the lens 18 and the diffuser plate 20, which makes the illumination more uniform across the fundus (Ef) of eye 100. The ring aperture plate 16 is conjugate with a position between the pupil (Ep) and the cornea of the eye 100 through the relay lenses 15, 13, and 6, the perforated mirror 2, and the ocular lens 1. The crystalline lens diaphragm 14 is conjugate with the back surface of the crystalline lens (Ecl) of eye 100 through relay lenses 13 and 6, and the ocular lens 1. Also, in this optical setup, the cornea ring aperture 17 is conjugate with the cornea.

FIGS. 9A and 9B show an embodiment of index optical subassembly 9. As shown in FIGS. 9A and 9B, index optical subassembly 9 includes a translucent plate 9e on which a pattern of thin light-blocking material is fixed. The light blocking material includes fixation targets 9d. Additionally, fixation target 9d at the center of translucent plate 9e forms a focus index generating rectangle 9c. As shown in FIG. 9B, a bi-prism 9b can be fixed adjacent to focus index generating slit 9c.

As shown in FIG. 9A, focus index generating rectangle 9c can be a slit opening surrounded by a light-blocking central disk and multiple fixation targets 9d, which can be shown as black dots or small openings as in FIGS. 9A and 9B. These fixation targets can be used to stabilize the eye under examination by drawing the patient's attention to one of these fixation targets. The axial position of these fixation targets 9d relative to that of the slit 9c can be adjusted to compensate for the field curvature and the index of refraction of the bi-prism 9b so that images of both the fixation targets and the focus index are at focus together at the fundus (Ef) of eye 100. In some embodiments, the bi-prism 9b is attached on top of the slit 9c and deflects the incident beam into two opposite directions.

FIG. 9A shows an exemplary top view of the translucent plate 9e with the slit 9c and the fixation targets 9d. FIG. 9B shows a side view of the translucent plate 9e in FIG. 9A with the bi-prism 9b mounted on top of the slit 9c. Referring back to FIG. 7 and FIG. 8, the focus index optics assembly 9 is held in position by a mechanical housing structure 9a which is fastened on the shaft of the solenoid 8, in FIG. 8 for example, so that the focus index optics assembly can be flipped in and out of the fundus illumination path correspondingly when the operator switches between the observation mode and the color image capturing mode.

In accordance with FIG. 8, when the operator adjusts the focus of the system, the combination of the focus index optical assembly 9 and the solenoid 8 mounted on a translation stage, can be moved longitudinally along the optical axis together with the movement of the sensor 5 at different rates, such as with a CAM wheel structure or a gear system. This approach eliminates the need for a focusing lens since the position of the sensor is adjusted for focusing.

Since the slit 9c is conjugate with the fundus Ef of eye 100, the split-bar pattern is superimposed onto the fundus image captured by the sensor 5 through the ocular lens 1, the central opening of the perforated mirror 2, the aperture stop 3, the lens slider 22, and the relay lens system 4. The split-bar pattern 30 and the stage of focus can then be displayed on a display device so that the operator can observe and adjust the focus to align the two halves of the split-bar (the focus index 30) for focusing (see FIGS. 6A-6C).

In some embodiments, sensor 5 can be a dual-band sensor that can capture both color and NIR images. An example of this type of sensor can be constructed by removing the IR cut filter of a typical solid-state sensor, such as a color CMOS or a CCD sensor where the silicon material is sensitive to visible wavelength band and NIR wavelength band up to around 1,000 nm. This approach has the advantage of using only one sensor to serve both the observation mode (using NIR light) and the image capturing mode (using visible light). Removing the IR cut filter has the advantage of letting the white LED illumination penetrate deeper into the choroid area of eye 100. However, such an approach can also blur the color image slightly due to chromatic aberration. In some embodiments, this disadvantage is overcome by attaching a small IR cut filter 23, such as one used for a typical cell phone camera, in front of each white LED 19a.

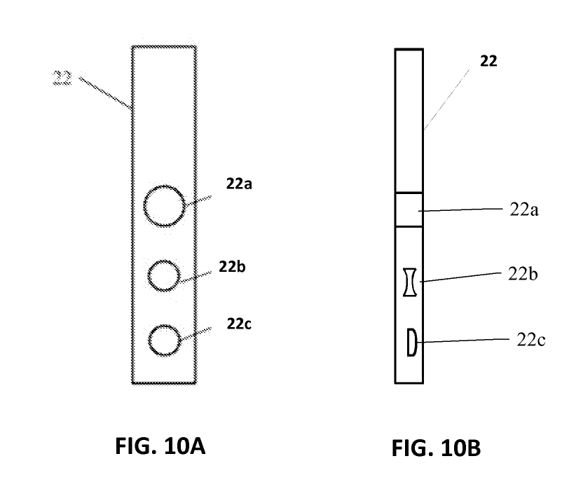

According to some embodiments as shown in FIG. 7 and FIG. 8, a first lens slider 22, which is further illustrated in FIGS. 10A-B, can be inserted in the imaging path. As is shown in FIGS. 10A and 10B, lens slider 22 can include mountings for lenses 22a, 22b, and 22c. As discussed above, lens 22a can be an opening; lens 22b can be a negative DC lens; and lens 22c can be a positive DC lens. Slider 22 can be positioned into the imaging path such that the imaging beam passes through one of lenses 22a, 22b, or 22c.

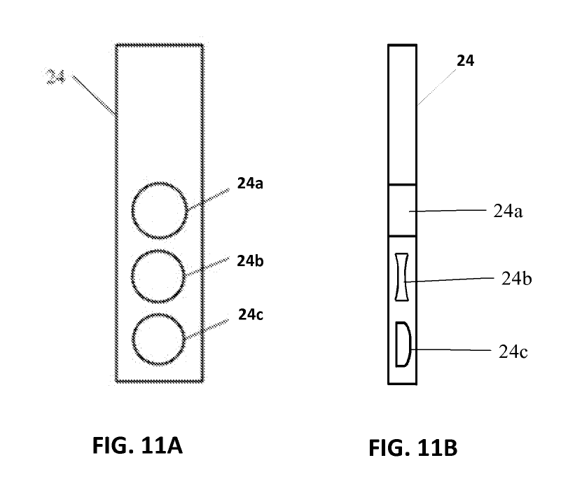

A second lens slider 24, which is positioned in the illumination path, is illustrated in FIGS. 11A-B. As shown in FIGS. 11A and 11B, lens slider 24 can include mounting for lenses 24a, 24b, and 24c. As discussed above, lens 24a can be an opening; lens 24b can be a negative DC lens; and lens 24c can be a positive DC lens. Slider 24 can be positioned in the illumination path such that the illumination beam passes through one of lenses 24a, 24b, or 24c.

Lens slider 22 and lens slider 24 can be positioned to achieve adequate focus range for different eye conditions during focusing and image acquisition mode. In some embodiments, the direction of movement for the two lens sliders 22, and 24 was shown in the plane of FIG. 7 and FIG. 8; in other embodiments the direction of travel can be along any other directions, such as transverse to the drawing plane.

For fundus imaging of a patient's eye with minor refractive error, the operator can move the lens sliders 22 and 24 to its first position, 22a and 24a, which are open holes, as shown in FIG. 10 and FIG. 11. For a patient with severe myopia, the lens sliders 22 and 24 can be moved to the second position, 22b and 24b, which are negative lenses, for diopter compensation. For a patient with severe presbyopia or hyperopia, the sliders 22 and 24 can be moved to the third position, 22c and 24c, which are positive lenses, for diopter correction. Note that the arrangement of the order of the DC lenses disclosed is only exemplary and it can be arranged in any order as long as the sliders 22 and 24 are matched with each other. The insertion of the lens sliders for diopter correction can be done manually or automatically. Also, they can be inserted into the optical path separately or simultaneously.

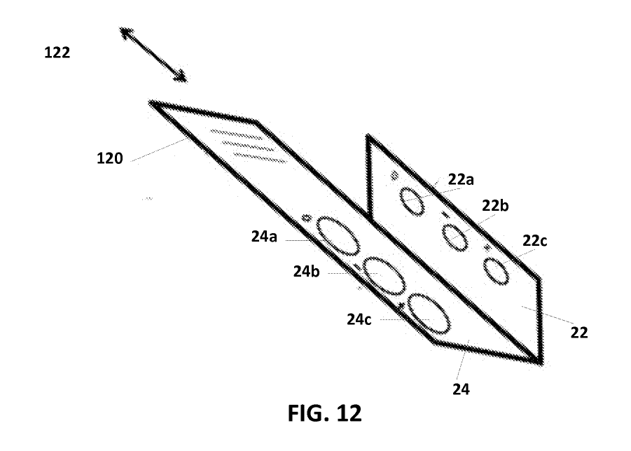

In some embodiment, lens slider 22 and lens slider 24 can be integrally formed into a v-shaped slider 122 that links the movement of lens sliders 22 and 24 as a single piece. Such an arrangement is illustrated in FIG. 12, where the label “0” indicates the first position with open hole lenses 22a and 24a, “−” indicates a negative lens such as lenses 22b and 24b, and “+” indicates a positive lens such as 22c and 24c. A handle 120 can be used to move the slider assembly 122 in and out manually by hand or automatically by a mechanical actuator. For the case when each of the lens sliders 22 and 24 is inserted separately, two position sensors can be inserted in the imaging and the illumination path to detect the position of each lens slider and to ensure the matched lenses are used. In some embodiments, an audio signal or a visible signal can be generated to alert operator if an incorrect matching of lens slider 22 and 24 is used. In other embodiments, the signal from the position sensor of the lens slider can also be used to guide the movement of the dot plate 21 (longitudinally) to the predetermined position so that reflection for the ocular lens can be eliminated for all three positions of the DC lens sliders 22 and 24.

In some embodiments, a variable focal-length lens can be used in place of the lens sliders 22 and 24 illustrated in FIG. 7 and FIG. 8. For example, a “focus tunable lens” (available from Optotune, Inc., Switzerland) either manually or electrically tunable, or a “liquid lens” (available from Varioptic, France) can be used in place of lens sliders 22 and 24.

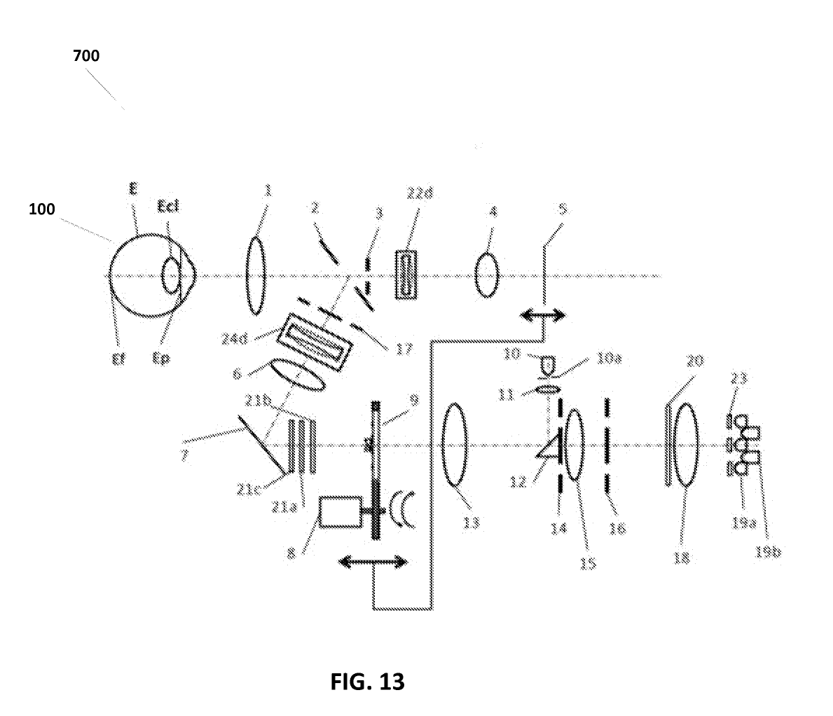

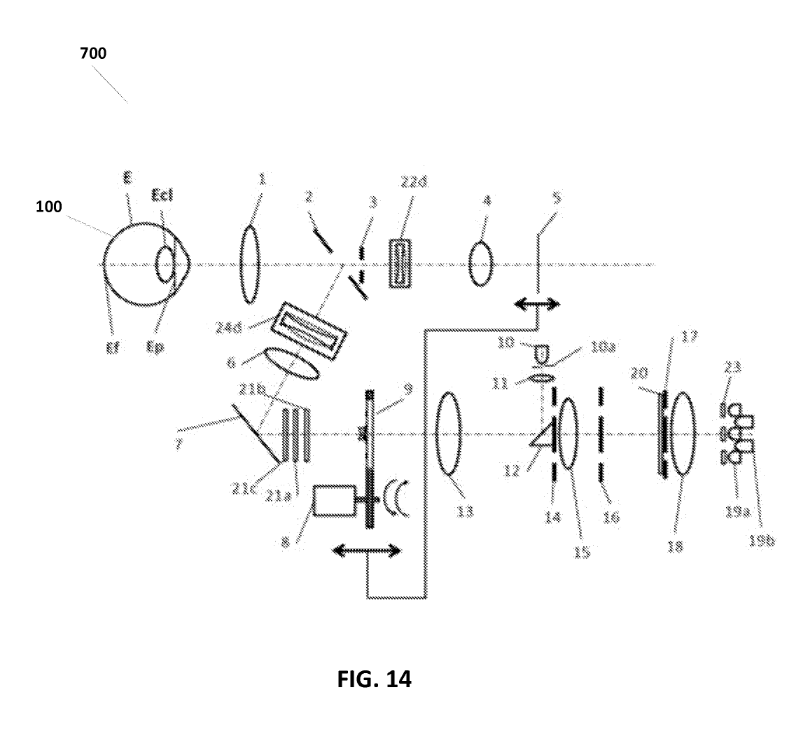

In general, any device capable of providing lenses in the illumination beam and matching lenses in the imaging beam can be used. In some embodiments, a smaller form factor liquid lens 22d can be used in place of the DC lens 22a to 22c in the imaging path due to a smaller beam size and a larger form factor tunable lens 24d can be used in place of the DC lens 24a to 24c in the illumination path, as shown in FIG. 13. In some embodiment, as shown in FIG. 14, it is advantageous to have more room to accommodate the DC lens slider 24 or the tunable lens assembly 24d, the cornea diaphragm 17 can be moved to another location which is also conjugated with the cornea. For example, the cornea diaphragm can be located immediately behind the diffuser plate 20 as shown in FIG. 14. A controller (not shown) commonly incorporated in a fundus camera 700 can be employed to generate a plurality of discrete voltage values to shift the diopter scale of the electrically tunable lens 22d, and 24d to match the required diopter correction value without the need for any translational mechanical movement needed in the DC lens slider 22 and 24. This approach can reduce the number of moving parts in the fundus camera and less moving parts is commonly known in the art to help improve reliability of the system. In some embodiments, a manually tunable lens can be used and its adjustable ring can be rotated to a plurality of angular positions to achieve the required diopter correction. The diopter adjustment can be achieved, either manually or automatically, by a mechanical linkage controlled by the controller which synchronizes the adjustment with the positional change of the DC lens slider 22 in the imaging path. In other embodiments, a plano-convex tunable lens can also be used. In this case, the last element of the lens system 6 can be combined with the plano-convex tunable lens so that the lens combination can produce the required plurality of discrete values in diopter scale to achieve proper diopter compensations. To further reduce the number of moving parts, according to some embodiments of the present invention, the axially movable dot plate 21 can be replaced by a set of three dot plates 21a-c with each one located at a preset position corresponding to each diopter setting of the DC lens, 24a to 24c of FIG. 7 and 8 or 24d of FIGS. 13, 14, and 15 to eliminate ocular lens reflection from any one of the three settings of the DC lens in the illumination path.

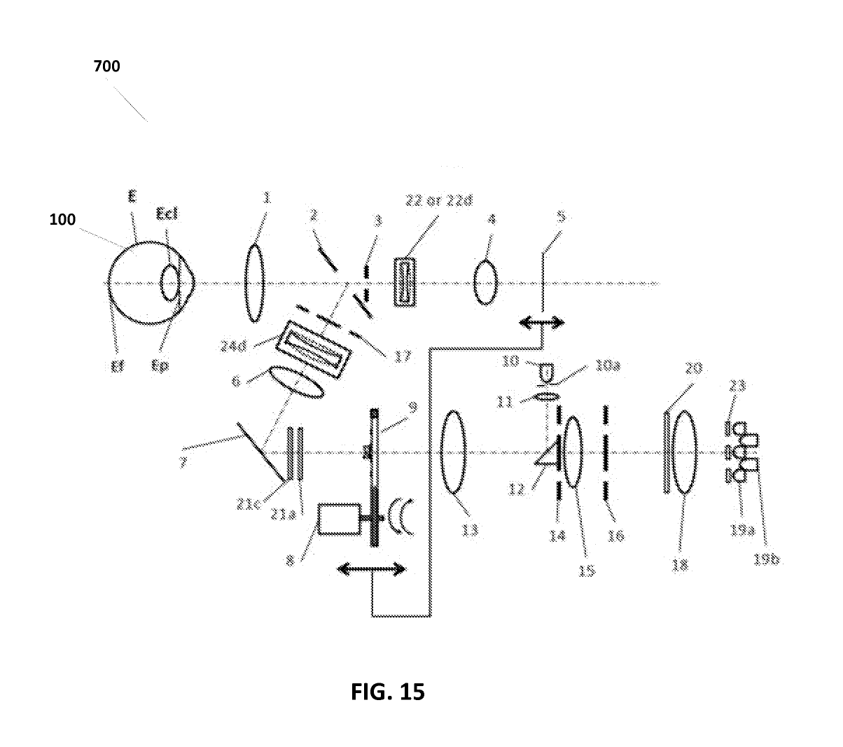

FIG. 15 shows an exemplary optical schematic of the fundus camera 700 according to some embodiments of the present invention. The dot plate 21b corresponding to the negative DC lens 24b, or the negative power of the tunable lens 24d, can be removed from the illumination path to increase the travel range of the focus index assembly 9. The ocular lens reflection during the observation mode can still be minimized in this embodiment since most of the unwanted reflection is blocked by the black disc around the slit 9c (see FIG. 9). In this embodiment using the tunable lens 24d, after focus adjustment is done by aligning the split-bar 30, the DC lens 24d in the illumination path is quickly changed back to zero diopter before capturing the color fundus image. In another words, the ocular lens reflection is still eliminated by dot plate 21a as if no DC lens is used in the capturing mode although a negative power lens was used during focus adjustment. In some embodiments, in the imaging path, the DC lens can be either the DC lens slider 22, or the tunable lens 22d.

The above examples are provided in order to demonstrate and further illustrate certain embodiments and aspects of the present invention and are not to be construed as limiting the scope thereof. In the description above, reference is made primarily to the eye as the object. This has to be understood as merely a way to help the description and not as a restriction of the application of the present invention. As such, where the term “eye” is used, a more general transparent and scattering object or organ may be sought instead. Although various embodiments that incorporate the teachings of the present invention have been illustrated and described in detail herein, a person of ordinary skill in the art can readily device other various embodiments that incorporate the teachings of this subject invention.

We claim:

1. An ophthalmic imaging apparatus, comprising:
an illumination path;
an imaging path;
a sensor in the imaging path;
a focus index optical assembly in the illumination path;
a first diopter compensator in the illumination path; and
a second diopter compensator in the imaging path, the first diopter compensator and the second diopter compensator cooperating to provide a focused image at the sensor.

2. The apparatus of claim 1, further comprising a focus lens in the imaging path, the focus lens coupled to move simultaneously with the focus index optical assembly and cooperating with the first and the second diopter compensator to provide a focused image at the sensor.

3. The apparatus of claim 1, wherein the focus index optical assembly is coupled to move simultaneously with the sensor and cooperating with the first and the second diopter compensator to provide a focused image at the sensor.

4. The apparatus of claim 1, further comprising at least one dot plate to eliminate surface reflections of an eye.

5. The apparatus of claim 4, wherein the at least one dot plates are placed in fixed positions corresponding to a setting of the second diopter compensator.

6. The apparatus of claim 4, wherein the at least one dot plate is moved to positions corresponding to a setting of the second diopter compensator.

7. The apparatus of claim 1, further comprising a ring aperture, the ring aperture is placed in a plane conjugate to a position between the pupil and the cornea of an eye in the illumination path.

8. The apparatus of claim 1, wherein the first diopter compensator and the second diopter compensator are neutral.

9. The apparatus of claim 1, wherein the first diopter compensator and the second diopter compensator are negative DC lenses.

10. The apparatus of claim 1, wherein the first diopter compensator and the second diopter compensator are positive DC lenses.

11. The apparatus of claim 1, wherein the first diopter compensator and the second diopter compensator are lens sliders.

12. The apparatus of claim 11, wherein the lens sliders are integrally formed into a v-shaped assembly.

13. The apparatus of claim 1, wherein the first diopter compensator and the second diopter compensator are tunable lenses.

14. The apparatus of claim 13, wherein the tunable lenses can be manually, automatically, mechanically, electronically tunable lenses, liquid lenses, or plano-convex lenses.

15. The apparatus of claim 1, wherein the sensor is a multi-band sensor.

16. A method of providing an ophthalmic image, comprising:
providing an illumination path with a focus index optical assembly and a first diopter compensator;
providing an imaging path with a sensor and a second diopter compensator; and
adjusting the first diopter compensator and the second diopter compensator to provide focus according to a focus index from the focus index optical assembly.

17. The method of claim 16, wherein adjusting the first diopter compensator and the second diopter compensator includes positioning a first slider in the illumination path and a second slider in the imaging path.
